(19) United States
(12) Patent Application Publication
(10) Pub. No.: US 2026/0194503 A1
(43) Pub. Date: Jul. 9, 2026

(54) Gas Detection System with Enhanced Selectivity

(71) Applicant: INDUSTRY-ACADEMIC COOPERATION FOUNDATION, YONSEI UNIVERSITY, Seoul (KR)

(72) Inventors: Kyu Hyoung LEE, Seoul (KR); Min Young KIM, Seoul (KR); Chul Oh PARK, Seoul (KR); Ha Eun CHOA, Seoul (KR)

(21) Appl. No.: 19/437,252

(22) Filed: Dec. 30, 2025

(30) Foreign Application Priority Data
Jan. 6, 2025 (KR) 10-2025-0001533

(51) Int. Cl. G01N 33/00

(52) U.S. Cl. CPC G01N 33/0016; G01N 33/0006; G01N 33/0018; G01N 33/0019; G01N 33/0034

(57) ABSTRACT

The present disclosure relates to a gas sensing method with enhanced selectivity. Specifically, the present disclosure relates to a gas sensing method for more accurately distinguishing various types of gases through a process of analyzing a correlation of two or more kinetic or static parameters from a temperature-dependent resistance change of a gas sensor according to two or more temperature conditions by changing the temperature of a captured external gas via a temperature control device.

### CROSS-REFERENCE TO RELATED APPLICATION

This application claims priority to Korean Patent Application No. 10-2025-0001533 filed in the Korean Intellectual Property Office on Jan. 6, 2025, the disclosure of which is incorporated by reference herein in its entirety.

### TECHNICAL FIELD

The present disclosure relates to a gas sensing method with enhanced selectivity for a gas, and more particularly, to a gas sensing method for more accurately distinguishing various types of gases through a process of analyzing a correlation between two or more kinetic or static parameters using a temperature-dependent resistance change of a gas sensor under two or more temperature conditions by changing a temperature of a gas.

### DISCUSSION OF RELATED ART

Gas sensors have been used in a wide range of fields such as chemistry, pharmacy, environment, and medicine, and it is predicted that further research will be conducted in the future. Technology for detecting harmful substances and pollutants in the air in real time is indispensable for maintaining a favorable living and working environment.

Currently, electrochemical gas sensors and semiconductor-type gas sensors are being used to effectively detect low concentrations of harmful gases. Among them, semiconducting metal oxide gas sensor devices in particular, despite having disadvantages of lower sensitivity and reliability compared to electrochemical-based gas sensors, have been continuously researched and developed with various dimensions, compositions, morphologies, microstructures, and functionalizations because they have advantages such as (i) low manufacturing cost, (ii) small size, (iii) a relatively simple operating principle, (iv) a wide variety, (v) excellent compatibility, and (vi) ease of controlling various compositions, morphologies, and microstructures.

Conventional gas sensors have a limitation of difficulty in clearly distinguishing a type of gas due to low selectivity among various gas components. In general, when reactivity is high, a problem of decreased selectivity among various gases arises as reactivity increases for all gases.

Accordingly, the present inventors have developed a new gas sensing method to solve this problem of low selectivity in conventional gas sensors.

### SUMMARY

An object of the present disclosure, devised to solve the disadvantage of low selectivity in conventional gas sensors, is to provide a gas sensing method for more accurately distinguishing various types of gases through a process of analyzing a correlation between two or more kinetic or static parameters using a temperature-dependent resistance change of a gas sensor under two or more temperature conditions by changing a temperature of a captured external gas through temperature control.

To achieve the above object, the present disclosure provides a gas sensing method including: capturing an external gas; measuring a resistance value (Rₒ) of a gas sensor under a base gas condition to stabilize the gas sensor; measuring resistance values (R_{g}) of the gas sensor under two or more temperature conditions with different temperatures by changing the temperature of the external gas; after calculating ratios (R_{g}/Rₒ) of the resistance value of the gas sensor under the base gas condition to the resistance values of the gas sensor under the two or more temperature conditions with different temperatures, calculating two or more kinetic parameters or static parameters from the ratios (R_{g}/Rₒ); and determining a type of the gas by comparing the correlation of the two or more kinetic or static parameters calculated in the operation iv) with a standard correlation of two or more kinetic or static parameters.

The gas sensing method of the present disclosure allows for easy and accurate distinction of gas types through a process of analyzing a correlation of two or more kinetic parameters or static parameters using a temperature-dependent resistance change of a gas sensor. Furthermore, in the gas sensing method of the present disclosure, the correlation of the kinetic or static parameters may be visualized using a scatter plot. Therefore, the gas sensing method of the present disclosure may more easily and accurately distinguish a type of gas by representing the correlation of kinetic or static parameters for a target external gas as a scatter plot and then comparatively analyzing the scatter plot with a standard scatter plot for each gas.

Furthermore, the gas sensing method of the present disclosure may measure resistance values of a gas sensor for more diverse conditions or test samples in which one or more of concentration, humidity, and light absorbance are additionally changed in addition to the temperature of the gas. Therefore, the gas sensing method of the present disclosure may distinguish various types of gases more accurately compared to a case where only the aforementioned temperature change is applied.

Furthermore, the gas sensing method of the present disclosure may enhance gas selectivity by more clearly distinguishing various gases, and provides more efficient detection performance compared to conventional gas sensors. In particular, fast and accurate identification of hazardous gases is enabled, which may provide practical advantages in industrial sites and for personal safety devices.

Furthermore, because a multi-array system may be applied to the gas sensing method of the present disclosure, a plurality of gas sensors may be arranged in parallel to simultaneously detect and analyze multiple types of gases. Such a multi-array application method complements the characteristics and selectivity of individual sensors, and by comprehensively processing data from the plurality of sensors, more accurate and precise gas identification is possible, and the stability and reliability of the entire system may be enhanced.

Furthermore, the gas sensing method of the present disclosure may be miniaturized, and through this, the gas sensing system may be easily integrated into various everyday electronic devices such as smartwatches, attachable sensors, and portable electronic devices. Thanks to these characteristics, the present disclosure provides a user-friendly and economical system and may have high commercial value in the field of gas detection and analysis.

### BRIEF DESCRIPTION OF THE DRAWINGS

FIG. 1 is a schematic diagram of a gas sensor system including a gas capturing unit, a gas temperature control unit, a gas sensor unit, and a data analysis unit, according to an exemplary embodiment of the present disclosure.

FIG. 2 is a schematic diagram of a gas sensor system including a gas capturing unit, a gas temperature control unit, a test sample storage unit, a gas sensor unit, and a data analysis unit, according to an exemplary embodiment of the present disclosure.

FIG. 3 is a schematic diagram of a gas sensor system including a gas capturing unit, a gas sensor unit, and a data analysis unit, wherein the gas sensor system includes a separate gas temperature control device inside the gas sensor unit, according to an exemplary embodiment of the present disclosure.

FIG. 4 is a graph showing results measured for NH₃, H₂, and Acetone gases at a temperature of 300° C. using ZnO, Au—ZnO, and Pd—ZnO sensors, according to an exemplary embodiment of the present disclosure. The graph shows the correlation between K and LLOD, the correlation between β (Beta) and LLOD, and the correlation between n and LLOD.

FIG. 5 is a graph showing results measured for NH₃, H₂, and Acetone gases at a temperature of 350° C. using ZnO, Au—ZnO, and Pd—ZnO sensors, according to an exemplary embodiment of the present disclosure. The graph shows the correlation between K and LLOD, the correlation between β (Beta) and LLOD, and the correlation between n and LLOD.

FIG. 6 is a graph showing results measured for NH₃, H₂, and Acetone gases at a temperature of 400° C. using ZnO, Au—ZnO, and Pd—ZnO sensors, according to an exemplary embodiment of the present disclosure. The graph shows the correlation between K and LLOD, the correlation between β (Beta) and LLOD, and the correlation between n and LLOD.

FIG. 7 is a graph showing results measured for NH₃, H₂, and Acetone gases at a temperature of 450° C. using ZnO, Au—ZnO, and Pd—ZnO sensors, according to an exemplary embodiment of the present disclosure. The graph shows the correlation between K and LLOD, the correlation between β (Beta) and LLOD, and the correlation between n and LLOD.

### DETAILED DESCRIPTION

Hereinafter, the present disclosure will be described in detail. In describing the present disclosure, detailed descriptions of related known configurations or functions may be omitted.

Terms or words used in this specification and the claims should not be interpreted as being limited to common or dictionary meanings, but should be interpreted with meanings and concepts consistent with the technical aspects of the present disclosure.

The embodiments described in this specification and the configurations illustrated in the drawings are preferred embodiments of the present disclosure, and do not represent all of the technical ideas of the present disclosure, so it should be understood that there may be various equivalents and modifications capable of replacing them at the time of filing of the present application.

The present disclosure provides a gas sensing method including: capturing an external gas; measuring a resistance value (Rₒ) of a gas sensor under a base gas condition to stabilize the gas sensor; measuring resistance values (R_{g}) of the gas sensor under two or more temperature conditions with different temperatures by changing the temperature of the external gas; after calculating ratios (R_{g}/Rₒ) of the resistance value of the gas sensor under the base gas condition to the resistance values of the gas sensor under the two or more temperature conditions with different temperatures, calculating two or more kinetic parameters or static parameters from the ratios (R_{g}/Rₒ); and determining a type of the gas by comparing the correlation of the two or more kinetic or static parameters calculated in the operation iv) with a standard correlation of two or more kinetic or static parameters.

In the method, the capturing of the external gas in the operation i) may use both active capturing using a pump and passive capturing naturally capturing gas using diffusion or the like.

The external gas may be captured using various gas capturing devices known in the art.

In one embodiment, the gas capturing device may include a container for storing the captured gas, and may additionally include at least one selected from the group consisting of a filter for filtering out large particles such as dust, an adsorbent for selectively adsorbing a specific gas, a moisture removal filtration device for adsorbing moisture to minimize the influence of moisture in the air, a pump for moving the gas to a capturing container, a diffusion tube for diffusing the gas, a vacuum device for creating a vacuum state, and a device for analyzing and monitoring the external gas.

In the method, the base gas in the operation ii) may be air, an inert gas such as nitrogen or argon, or a mixed gas of oxygen and an inert gas, and a gas of the same type as or a different type from the dilution gas may be used.

The operation ii) may be performed to measure R₀, which is a resistance value of the gas sensor in a reference state.

In the method, changing the temperature of the captured external gas in the operation iii) may be performed by heating or cooling the captured external gas using a temperature control unit.

The temperature change may be prepared at various desired temperatures using a variety of gas temperature control devices known in the art (e.g., a heater or a cooler). In this case, the temperature control unit may be provided external to the gas sensor as a separate device, or may be integrated inside the gas sensor, such as in a Micro Electro Mechanical System (MEMS).

In the method, the temperature change in the operation iii) may be generated within a chamber including the gas sensor, or test samples with different temperatures may be prepared in advance before being exposed to the gas sensor, stored in a storage container, and then moved to a chamber including the gas sensor.

In one embodiment, when the temperature change is generated within a chamber including the gas sensor, a gas capturing device is connected to a chamber including the gas sensor, and a temperature of a gas captured in the chamber may be controlled through a gas temperature control device located inside or outside the chamber.

In one embodiment, when the temperature change is generated outside a chamber before exposure to the gas sensor, first, a gas capturing device and a gas temperature control device are respectively connected to a plurality of storage containers, and a temperature of a gas captured in each of the plurality of storage containers is controlled through the gas temperature control device to prepare test samples with different temperatures. Then, the test samples with different temperatures from the plurality of storage containers are moved to a chamber including the gas sensor to create various temperature conditions.

In the method, in the operation iii), in addition to temperature, one or more of concentration, humidity, or light absorbance may be changed to additionally create conditions with different concentrations, humidities, or light absorbances. Through this, by preparing test samples with more diverse combinations by changing conditions other than temperature and performing the gas sensing of the present disclosure, the gas may be distinguished more accurately as a result value from the change in other conditions is added, compared to distinguishing the gas by the result value from the temperature change. Consequently, selectivity for the gas may be enhanced.

The conditions with different concentrations, humidities, or light absorbances may be controlled using a concentration control device, a humidity control device, and a light source device, respectively. In this case, the concentration control device, the humidity control device, or the light source device may be provided external to the gas sensor as a separate device. Alternatively, the humidity control device or the light source device may be integrated inside the gas sensor, such as in a Micro Electro Mechanical System (MEMS).

As the concentration control device, a gas dilution device or a gas concentration device may be used, and these may prepare test samples with various concentrations by injecting a dilution medium (e.g., an inert gas such as air, nitrogen, or argon, or a mixed gas of oxygen and an inert gas) into the gas or by reducing a volume of the gas through compression.

In one embodiment, the gas dilution device may include a container for storing a dilution medium, a dilution gas injection unit for injecting the dilution medium into the captured external gas, and a plurality of containers for storing two or more test samples with changed concentrations after the dilution medium is injected into the captured external gas. Furthermore, the gas dilution device may additionally include at least one selected from the group consisting of a device for controlling a flow velocity of a gas (captured gas and diluted gas), a device for detecting a gas leak, a flow rate control device, and a real-time monitoring and control device.

In one embodiment, the gas concentration device may include a compressor for increasing a pressure of a gas by reducing a volume of the gas, and a plurality of containers for storing two or more test samples with changed concentrations after being concentrated. Furthermore, the gas concentration device may additionally include at least one selected from the group consisting of a device for measuring a pressure of the container, a pressure relief valve for maintaining a normal pressure value by discharging pressure, preventing the pressure from exceeding a specified value, a device for detecting overpressure and leakage, and a real-time monitoring and control device.

As the humidity control device, a moisture supply or a humidity generator may be used. These may prepare test samples with various gas humidities by treating the gas with different amounts of moisture supply.

As the light source device, a UV light-emitting element, a UV-LED light-emitting element, or a visible light wavelength LED light-emitting element may be used. These may prepare test samples with various gas light absorbances by treating the gas with different light wavelengths or intensities.

In the method, in the operation ii) or operation iii), when the gas sensor is exposed to a base gas or an external gas, a relative change in a resistance value for each gas temperature may be calculated by measuring a resistance value of the sensor.

The gas sensor may be a semiconductor-type gas sensor based on a metal oxide material (e.g., a metal oxide selected from the group consisting of ZnO, SnO₂, In₂, O₃, WO₃, CuO, and NiO) or a metal-metal oxide material (e.g., a material in which metal particles selected from the group consisting of platinum (Pt), palladium (Pd), silver (Ag), and gold (Au) are attached to the metal oxide), having a resistance changing in response to a reaction with a specific gas.

In the method, in the operation ii) or operation iii), the gas sensor may be included in a chamber, and multiple gas sensing may be performed by including a plurality of gas sensors, each detecting a different type of gas, in the chamber.

In one embodiment, the gas sensor may further include, in addition to the material whose resistance changes by reacting with a specific gas, at least one of a power supply, a source measurement unit (SMU) for precisely measuring a source voltage or current, or device connection accessories.

In one embodiment, the gas sensor may include at least one of the temperature control device, the concentration control device, the humidity control device, or the light source device therein to measure a resistance value by changing a temperature, concentration, humidity, or light absorbance of a gas.

In the method, in the operation iv), the kinetic or static parameters may be directly calculated from a change in the temperature-dependent resistance value of the gas sensor using calculation formulas known in the art, such as a power law model.

Furthermore, the kinetic or static parameters may be calculated via a data processing program executed using output data of the gas sensor (e.g., time-concentration data of resistance/current/voltage signals) as input. The data processing program may be one or more programs selected from the group including OriginPro, MATLAB, Python, Lab VIEW, Excel, and R. The program may, for example, extract a regression, curve fitting, slope/exponent value of Equation 1 below.

The kinetic parameter may be any one selected from the group consisting of K, β (Beta), LLOD (lower limit of detection), Eb (Energy barrier), and n (Power law Exponent), and the static parameter may be any one selected from the group consisting of t90, T90, Carrier Concentration, and Oxygen Vacancy.

The kinetic parameter or the static parameter may be defined as follows.
K: K is a reaction rate coefficient value derived by mathematically analyzing a resistance response behavior of the gas sensor. The K is a value calculated by an equation, is related to the adsorption and desorption of oxygen, and is a kinetic parameter related to a rate at which an external gas reacts with oxygen and a material surface.
β (beta): β is an exponent value representing a relationship between sensor response and gas concentration. The β is a value calculated by an equation and is a kinetic parameter related to an interaction between an external gas and an ionized oxygen species present on a material surface.
LLOD: LLOD is a minimum gas concentration value at which a gas may be detected. The LLOD is a gas concentration at which an external gas may be detected and is a kinetic parameter obtained from K and β (beta), which are calculated by an equation.
T90: T90 is a gas detection speed, being a time taken for a sensor signal to reach 90% of its final response value after an injection of an external gas. The T90 is a static parameter representing a response time and is a value indicating how quickly an external gas is detected.
t90: 190 is a recovery characteristic of the sensor, being a time taken for a sensor signal to recover to 90% of its initial state after a gas is removed. The t90 is a static parameter representing a recovery time and is a value indicating how quickly an external gas is recovered.
Carrier Concentration: Carrier Concentration is a density of free charges (electrons or holes) contributing to electrical conduction within a semiconductor material. The Carrier Concentration is a static parameter related to various kinetic parameters and static parameters.
Oxygen vacancy: An Oxygen vacancy is a point defect formed by an absence of an oxygen atom within an oxide crystal structure. The Oxygen vacancy is a static parameter related to various kinetic parameters and static parameters.
Eb: Eb is a height of an energy barrier in an electron depletion layer formed by oxygen adsorbed on a surface of a material. The Eb is a kinetic parameter.
n: n refers to a form of charged oxygen created when adsorbed oxygen molecules on a solid surface or interface capture or release electrons. The n is a value determining a type of ionized oxygen ion species present on a material surface and is a kinetic parameter.

In one embodiment, the K, β (Beta), and LLOD may be calculated by Equation 1 below.

$$Sensing response ( S ) = \frac{{R}_{g}}{{R}_{a}} = K \cdot {C}^{β} \qquad Equation 1$$

Here,
Rg: electrical resistance when exposed to an analyte;
Ra: electrical resistance when exposed to dry air; and
C: concentration of the analyte.

In one embodiment, the Eb may be calculated by Equation 2 below.

$${R}_{e} = \frac{{R}_{g}}{{R}_{0}} = \frac{{N}_{d}}{{n}_{s}} = exp ( \frac{{qV}_{s}}{kT} ) \qquad Equation 2$$

$$qVs = {E}_{b} = \frac{{q}^{2}}{2 ε} \frac{{n}_{s}^{2}}{{N}_{o}} = \frac{{q}^{2}}{2 ε} {N}_{o} {W}^{2} = \frac{{k}_{B} T}{2} \frac{{W}^{2}}{{λ}_{D}^{2}}$$

Here,
Rg: resistance in a gas atmosphere;
Ro: resistance in a flat band state;
Re: reduced sensor resistance;
Vs: potential energy of an electron;
q: charge of a coulomb;
ε: dielectric constant;
Ns: surface charge carrier concentration;
No: bulk carrier concentration; and
Nd: donor density.

In the method, the operation v) is to distinguish a specific type of gas by comparing the correlation of two or more kinetic or static parameters calculated in the operation iv) with a standard correlation of two or more kinetic or static parameters pre-established for a specific type of gas.

In the operation v), the correlation of the two or more kinetic or static parameters includes the correlation among all of the two or more kinetic or static parameters, and in particular, may be one or more selected from the group consisting of the correlation between K and LLOD, the correlation between β (Beta) and LLOD, and the correlation between n and LLOD.

In the operation v), the standard correlation of the kinetic or static parameters may be a databased version calculated by repeating the processes of i) to iv) according to the present disclosure multiple times for the target gas.

In the operation v), the correlation of the two or more kinetic or static parameters may be visualized as a scatter plot, which may then be compared with a standard scatter plot visualizing the standard correlation of two or more kinetic or static parameters.

In the present disclosure, a mechanism in which an electrical resistance value changes according to properties of a specific material of a gas sensor is utilized, and a mechanism in which a reaction occurs on a surface of the material is applied. In particular, a range of resistance change occurring when an external gas is introduced under identical environmental conditions was measured, and a quantitative value was calculated therefrom.

In an exemplary embodiment of the present disclosure, properties of a material of a gas sensor were confirmed to potentially affect reactivity and cause a difference in a change of an electrical resistance value. For example, for a specific material, a resistance value increases or decreases depending on an interaction with gas molecules, which is due to an electron transfer mechanism or ion adsorption characteristics on the material surface. Such a resistance change may vary depending on external environmental conditions, a type and temperature and concentration of a gas, and microstructural properties of the material. A range of resistance change when an external gas is introduced may be calculated by the following method. Under identical environmental conditions, a resistance value in a reference state is set as R₀, and a resistance value in a state where a gas is introduced is measured as R_{g}. A resistance change ratio may be defined by the following equation.

$$Δ R = \frac{{R}_{g} = {R}_{o}}{{R}_{o}} \times 100 ( \% ) \qquad Equation 3$$

It may be confirmed that a range of resistance change of the resistance value obtained through the above equation appears differently depending on material properties of the gas sensor, and through this, a sensitivity and reactivity of the material of the gas sensor may be evaluated.

In an exemplary embodiment of the present disclosure, a resistance value change according to a type of external gas, temperature, and concentration was experimentally confirmed for various materials of a gas sensor. For example, in a case where the gas sensor is a metal oxide material, a greater range of change in the resistance value was observed for a specific gas, which may be considered to show a different mechanism due to an electron transfer effect according to surface adsorption depending on material properties of the gas sensor.

Furthermore, the present disclosure provides a gas sensing system which may be used to perform the gas sensing method described above.

The gas sensing system is a gas sensing system for performing a gas sensing method in which temperature control of an external gas is performed within a chamber including the gas sensor, among the gas sensing methods according to the present disclosure.

The gas sensing system of the present disclosure includes: a gas capturing unit for capturing an external gas; a gas temperature control unit for changing a temperature of the captured external gas by heating or cooling the captured external gas; a gas sensor unit for measuring a resistance value (Rₒ) of a gas sensor under a base gas condition to stabilize the gas sensor, and resistance values (R_{g}) of the gas sensor under two or more temperature conditions by the gas temperature control unit; and a data analysis unit for comparatively analyzing a correlation of two or more kinetic or static parameters calculated from the resistance values of the gas sensor unit.

Here, the data analysis unit first calculates ratios (R_{g}/Rₒ) of a resistance value (Rₒ) of the gas sensor under the base gas condition to resistance values (R_{g}) of the gas sensor for the two or more temperature conditions with different temperatures from the resistance values of the gas sensor unit. Then, the data analysis unit calculates two or more kinetic or static parameters from the ratios (R_{g}/Rₒ). Then, the data analysis unit comparatively analyzes the correlation of the two or more kinetic or static parameters with a standard correlation of two or more kinetic or static parameters.

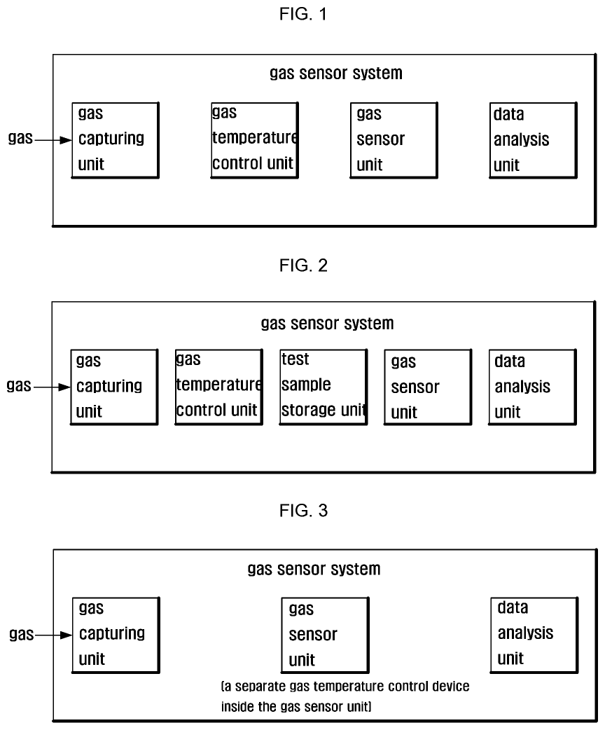

The gas sensing system may be configured as shown in the schematic diagrams described in FIG. 1 and FIG. 2.

FIG. 1 is a gas sensor system including a gas capturing unit, a gas temperature control unit, a gas sensor unit, and a data analysis unit.

FIG. 2 is a gas sensor system further including a test sample storage unit in the system of FIG. 1.

In the gas sensing system, the gas capturing unit is a means for capturing an external gas, and at this time, the capturing of the external gas may use both active capturing using a pump and passive capturing, naturally capturing gas using diffusion or the like.

The gas capturing unit may use various gas capturing devices known in the art.

In one embodiment, the gas capturing unit may consist of a container for storing the captured gas, and may additionally include at least one selected from the group consisting of a filter for filtering out large particles such as dust, an adsorbent for selectively adsorbing a specific gas, a moisture removal filtration device for adsorbing moisture to minimize the influence of moisture in the air, a pump for moving the gas to a capturing container, a diffusion tube for diffusing the gas, a vacuum device for creating a vacuum state, and a device for analyzing and monitoring the external gas.

In the gas sensing system, the gas temperature control unit may prepare conditions or test samples with different temperatures by heating or cooling the captured external gas using a temperature control device.

As the temperature control device, a heater or a cooler may be used.

In the gas sensing system, the gas sensor unit is a means for measuring a resistance value of the sensor when exposed to a base gas or an external gas, and through this, a relative resistance value change for each gas temperature may be calculated.

The gas sensor unit may include a gas sensor and a chamber including the gas sensor.

The temperature change may be performed using the gas temperature control unit. The temperature change may be generated within a chamber including the gas sensor, or a test sample with a changed temperature may be prepared in advance before being exposed to the gas sensor, stored in a storage container, and then moved to the chamber including the gas sensor.

In one embodiment, when the temperature change is generated within a chamber including the gas sensor, a gas capturing device is connected to the chamber including the gas sensor, and a temperature of a gas captured in the chamber may be controlled through a gas temperature control device located inside or outside the chamber.

In one embodiment, when the temperature change is generated outside a chamber before exposure to the gas sensor, first, a gas capturing device and a gas temperature control device are respectively connected to a plurality of storage containers, and a temperature of a gas captured in each of the plurality of storage containers is controlled through the gas temperature control device to prepare test samples with different temperatures. Then, the test samples with different temperatures may be moved from the plurality of storage containers to a chamber including the gas sensor.

In the gas sensor unit, the gas sensor may be a semiconductor-type gas sensor based on a metal oxide material (e.g., a metal oxide selected from the group consisting of ZnO, SnO₂, In₂, O₃, WO₃, CuO, and NiO) or a metal-metal oxide material (e.g., a material in which metal particles selected from the group consisting of platinum (Pt), palladium (Pd), silver (Ag), and gold (Au) are attached to the metal oxide), exhibiting a resistance change in response to a reaction with a specific gas.

The gas sensor unit may include one gas sensor in a chamber, and may include a plurality of gas sensors in the chamber to perform multiple gas sensing detecting different types of gases respectively.

The gas sensor unit may further include, in addition to the aforementioned material whose resistance changes by reacting with a specific gas, at least one of a power supply, a source measurement unit (SMU) for precisely measuring a source voltage or current, or device connection accessories.

The gas sensor unit may include at least one of a concentration control device, a humidity control device, or a light source device therein to measure a resistance value by changing a concentration, humidity, or light absorbance of a gas.

In one embodiment, the concentration control device may be a gas dilution device or a gas concentration device. The humidity control device may be a moisture supply or a humidity generator (e.g., an ultrasonic humidifier, a heating-type humidifier, a composite humidifier, etc.). The light source device may be a UV light-emitting element, a UV-LED light-emitting element, or a visible light wavelength LED light-emitting element.

The gas sensing system may include a test sample storage unit separate from the gas sensor unit.

The test sample storage unit may be a means for respectively storing two or more test samples with different temperatures prepared by the gas temperature control unit.

In one embodiment, the test sample storage unit may include two or more containers for storing two or more test samples, and may additionally include at least one selected from the group consisting of a flow velocity and flow rate control device, a device for detecting a gas leak, and a real-time monitoring and control device.

The gas sensing system may further include, externally and separate from the gas sensor unit, at least one of a concentration control device, a humidity control device, or a light source device.

The concentration control device, the humidity control device, or the light source device may additionally prepare test samples with different concentrations, humidities, or light absorbances by changing one or more of concentration, humidity, or light absorbance separately from temperature. Through this, by preparing test samples with more diverse combinations by changing conditions other than temperature and performing the gas sensor system of the present disclosure, the gas may be distinguished more accurately as a result value from the change in other conditions is added, compared to distinguishing the gas by the result value from the temperature change. Consequently, selectivity for the gas may be enhanced.

As the concentration control device, a gas dilution device or a gas concentration device may be used. These may prepare test samples with various concentrations by injecting a dilution medium into the gas or by reducing a volume of the gas through compression.

In one embodiment, the gas dilution device may include a container for storing a dilution medium, a dilution gas injection unit for injecting the dilution medium into the captured external gas, and a plurality of containers for storing two or more test samples with changed concentrations after the dilution medium is injected into the captured external gas. Furthermore, the gas dilution device may additionally include at least one selected from the group consisting of a device for controlling a flow velocity of a gas (captured gas and diluted gas), a device for detecting a gas leak, a flow rate control device, and a real-time monitoring and control device.

In one embodiment, the gas concentration device may include a compressor for increasing a pressure of a gas by reducing a volume of the gas, and a plurality of containers for storing two or more test samples with changed concentrations after being concentrated. Furthermore, the gas concentration device may additionally include at least one selected from the group consisting of a device for measuring a pressure of the container, a pressure relief valve for maintaining a normal pressure value by discharging pressure, preventing the pressure from exceeding a specified value, a device for detecting overpressure and leakage, and a real-time monitoring and control device.

As the humidity control device, a moisture supply or a humidity generator may be used. These may prepare test samples with various gas humidities by treating the gas with different amounts of moisture supply.

As the light source device, a UV light-emitting element, a UV-LED light-emitting element, or a visible light wavelength LED light-emitting element may be used. These may prepare test samples with various gas light absorbances by treating the gas with different light wavelengths or intensities.

In the gas sensing system, the data analysis unit first calculates two or more kinetic or static parameters from the resistance value calculated by the gas sensor unit using a calculation formula known in the art, such as a power law model. Then, the data analysis unit calculates two or more kinetic or static parameters from a change in the resistance value using a calculation formula known in the art, such as a power law model. Then, the data analysis unit compares the calculated correlation with a standard correlation of two or more kinetic or static parameters pre-established for a specific type of gas.

The data analysis unit may use one or more data processing programs selected from the group including OriginPro, MATLAB, Python, Lab VIEW, Excel, and R.

The kinetic parameter may be any one selected from the group consisting of K, β (Beta), LLOD (lower limit of detection), Eb (Energy barrier), and n (Power law Exponent), and the static parameter may be any one selected from the group consisting of t90, T90, Carrier Concentration, and Oxygen Vacancy.

The kinetic parameter or the static parameter may be defined as follows.
K: K is a reaction rate coefficient value derived by mathematically analyzing a resistance response behavior of the gas sensor. The K is a value calculated by an equation, is related to the adsorption and desorption of oxygen, and is a kinetic parameter related to a rate at which an external gas reacts with oxygen and a material surface.
β (beta): β is an exponent value representing a relationship between sensor response and gas concentration. The β is a value calculated by an equation and is a kinetic parameter related to an interaction between an external gas and an ionized oxygen species present on a material surface.
LLOD: LLOD is a minimum gas concentration value at which a gas may be detected. The LLOD is a gas concentration at which an external gas may be detected and is a kinetic parameter obtained from K and β (beta), which are calculated by an equation.
T90: T90 is a gas detection speed, being a time taken for a sensor signal to reach 90% of its final response value after an injection of an external gas. The T90 is a static parameter representing a response time and is a value indicating how quickly an external gas is detected.
t90: 190 is a recovery characteristic of the sensor, being a time taken for a sensor signal to recover to 90% of its initial state after a gas is removed. The t90 is a static parameter representing a recovery time and is a value indicating how quickly an external gas is recovered.
Carrier Concentration: Carrier Concentration is a density of free charges (electrons or holes) contributing to electrical conduction within a semiconductor material. The Carrier Concentration is a static parameter related to various kinetic parameters and static parameters.
Oxygen vacancy: An Oxygen vacancy is a point defect formed by an absence of an oxygen atom within an oxide crystal structure. The Oxygen vacancy is a static parameter related to various kinetic parameters and static parameters.
Eb: Eb is a height of an energy barrier in an electron depletion layer formed by oxygen adsorbed on a surface of a material. The Eb is a kinetic parameter.
n: n refers to a form of charged oxygen created when adsorbed oxygen molecules on a solid surface or interface capture or release electrons. The n is a value determining a type of ionized oxygen ion species present on a material surface and is a kinetic parameter.

In one embodiment, the K, β (Beta), and LLOD may be calculated by Equation 1 below.

$$Sensing response ( S ) = \frac{{R}_{g}}{{R}_{a}} = K \cdot {C}^{β} \qquad Equation 1$$

Here,
Rg: electrical resistance when exposed to an analyte;
Ra: electrical resistance when exposed to dry air; and
C: concentration of the analyte.

In one embodiment, the Eb may be calculated by Equation 2 below.

$${R}_{e} = \frac{{R}_{g}}{{R}_{0}} = \frac{{N}_{d}}{{n}_{s}} = exp ( \frac{{qV}_{s}}{kT} ) \qquad Equation 2$$

$$qVs = {E}_{b} = \frac{{q}^{2}}{2 ε} \frac{{n}_{s}^{2}}{{N}_{o}} = \frac{{q}^{2}}{2 ε} {N}_{o} {W}^{2} = \frac{{k}_{B} T}{2} \frac{{W}^{2}}{{λ}_{D}^{2}}$$

Here,
Rg: resistance in a gas atmosphere;
Ro: resistance in a flat band state;
Re: reduced sensor resistance;
Vs: potential energy of an electron;
q: charge of a coulomb;
ε: dielectric constant;
Ns: surface charge carrier concentration;
No: bulk carrier concentration; and
Nd: donor density.

The correlation of the two or more kinetic or static parameters includes the correlation among all of the two or more kinetic or static parameters, and in particular, may be one or more selected from the group consisting of the correlation between K and LLOD, the correlation between β (Beta) and LLOD, and the correlation between n and LLOD.

The standard correlation of the kinetic or static parameters may be a databased version of the correlation of two or more kinetic or static parameters of the target gas, which is calculated by repeatedly performing measurements through the gas sensor according to the present disclosure for the target gas.

The correlation of the two or more kinetic or static parameters may be visualized as a scatter plot, comparable with a standard scatter plot visualizing the standard correlation of two or more kinetic or static parameters.

Furthermore, the present disclosure provides another type of gas sensing system which may be used to perform the gas sensing method described above.

The gas sensing system is a gas sensing system for performing a gas sensing method in which a test sample with a different temperature is prepared in advance before being exposed to a gas sensor, among the gas sensing methods according to the present disclosure.

The gas sensing system includes:
a gas capturing unit for capturing an external gas;
a gas sensor unit including a gas temperature control device for changing a temperature of the captured external gas by heating or cooling the captured external gas, for measuring a resistance value (Rₒ) of a gas sensor under a base gas condition to stabilize the gas sensor, and resistance values (R_{g}) of the gas sensor under two or more temperature conditions by the gas temperature control device; and
a data analysis unit for comparatively analyzing a correlation of two or more kinetic or static parameters calculated from the resistance values of the gas sensor unit.

Here, the data analysis unit first calculates ratios (R_{g}/Rₒ) of a resistance value (Rₒ) of the gas sensor under the base gas condition to resistance values (R_{g}) of the gas sensor for the two or more temperature conditions with different temperatures from the resistance values of the gas sensor unit. Then, the data analysis unit calculates two or more kinetic or static parameters from the ratios (R_{g}/Rₒ). Then, the data analysis unit comparatively analyzes the correlation of the two or more kinetic or static parameters with a standard correlation of two or more kinetic or static parameters.

The gas sensing system may be configured as shown in the schematic diagram described in FIG. 3.

FIG. 3 is a gas sensor system composed of a gas capturing unit, a gas sensor unit, and a data analysis unit, wherein the gas sensor system includes a separate gas temperature control device inside the gas sensor unit.

In the gas sensing system, the gas capturing unit and the data analysis unit are the same as the descriptions for each component of the gas sensor system described above.

In the gas sensing system, the gas sensor unit includes a temperature control device therein, and thus may measure a resistance value under various temperature conditions by controlling a temperature of the sensor.

The gas sensor unit may further include, internally or externally, in addition to the temperature control device, at least one of a concentration control device, a humidity control device, or a light source device.

The concentration control device, the humidity control device, or the light source device may additionally prepare conditions or test samples with different concentrations, humidities, or light absorbances by changing one or more of concentration, humidity, or light absorbance separately from temperature. Through this, by preparing test samples with more diverse combinations by changing conditions other than temperature and performing the gas sensor system of the present disclosure, the gas may be distinguished more accurately as a result value from the change in other conditions is added, compared to distinguishing the gas by the result value from the temperature change. Consequently, selectivity for the gas may be enhanced.

Furthermore, the present disclosure provides an electronic device including the gas sensor system according to the present disclosure.

The gas sensor system may be miniaturized, and through this, it may be easily integrated into various everyday electronic devices such as smartwatches, attachable sensors, and portable electronic devices.

Hereinafter, the present disclosure will be described in more detail through examples.

These examples are only for explaining the present disclosure more specifically, and it is apparent to those of ordinary skill in the art that the scope of the present disclosure is not limited by these examples in accordance with the gist of the present disclosure.

### Example 1 Fabrication of Gas Sensor

For the gas sensor device, a previously manufactured MEMS system (Maimosi) device was purchased, components were assembled, and then gas sensing material was applied on top of the assembled gas sensor device by drop-casting or spraying in micrometer units to cover the electrodes. The resistance of the gas sensing material placed on the electrodes changes depending on the atmosphere, temperature, humidity, and wavelength and intensity of a light source within a gas sensing chamber. In this case, for the gas sensing materials (ZnO, Au—ZnO, and Pd—ZnO), ZnO, Au—ZnO, and Pd—ZnO, which are oxide semiconductors with excellent reactivity with surface oxygen ions and gases due to the formation of a surface defect structure through heat treatment, were used respectively.

### Example 2 Derivation of Graph Via Gas Sensor

An O₂/N₂ mixed gas with 21% O₂ and NH₃, Acetone, and H₂ gas cylinders were connected to a Mass Flow Controller (MFC) to control the gas atmosphere. For the gases, a concentration of 100 ppm/N₂ balance (Ilsung Gas) was ordered, a pressure and amount of the gas were confirmed by connecting a regulator to the gas cylinder, and a flow rate of the gas was controlled through the Mass Flow Controller (MFC) and introduced into the gas sensing chamber to form a gas atmosphere. Detailed concentrations and flow rates were controlled through a gas sensor tester (YNI-FLOW&PRESSURE CONTROLLER: MR-5000) and a built-in program. The temperatures of the gas sensing chamber and the gas sensor device were controlled through a temperature controller (MSTECH-Hot Chuck Controller: MST-1000H). The performance of the gas sensor was confirmed by simultaneously measuring voltage, current, and resistance through Keithley equipment (2450 Sourcemeter). The gas sensing performance was determined by measuring how much the resistance changed when a gas was introduced compared to the resistance when an O₂/N₂ mixed gas was introduced, and a material was determined to be a better gas sensing material if the amount of resistance change was large even at a low concentration.

During the gas sensing experiment, an O₂/N₂ mixed gas was introduced to the fabricated gas sensor to cause oxygen adsorption on the surface of the gas sensor material, and then the sensor was stabilized for more than 30 minutes until there was no change in the resistance of the sensor. Thereafter, a desired gas (NH₃, Acetone, H₂, etc.) was introduced, causing the gas sensor material and the gas to react, which changed the surface charge concentration of the material, and then the change in resistance over time was observed.

Ammonia (NH₃) gas was set in the order of 0.1 ppm, 0.25 ppm, 0.5 ppm, 1 ppm, 5 ppm, 25 ppm, and 50 ppm by concentration, and measurements were conducted for 15 minutes for each concentration.

Acetone gas was set in the order of 0.75 ppm, 2.5 ppm, 10 ppm, 50 ppm, 100 ppm, 250 ppm, and 275 ppm by concentration, and measurements were conducted for 15 minutes for each concentration.

Hydrogen (H₂) gas was set in the order of 0.75 ppm, 1 ppm, 2.5 ppm, 5 ppm, 10 ppm, 25 ppm, and 50 ppm by concentration, and measurements were conducted for 15 minutes for each concentration.

In the above experiment, a Mass Flow Controller (MFC) was used to precisely adjust a mixing ratio of a target gas and air to achieve each target concentration. Through this, a gas environment according to the target concentration was accurately implemented, and measurement data at each concentration was maintained for a fixed time to obtain reliable results.

The response of the gas sensor was obtained using the equation below.

### Equation

$$( Resistance when gas is introduced ) \div ( Resistance when {O}_{2} / {N}_{2} mixed gas is introduced )$$

After obtaining the response of the gas sensor, K, Beta, and LLOD were calculated through a power law calculation formula.

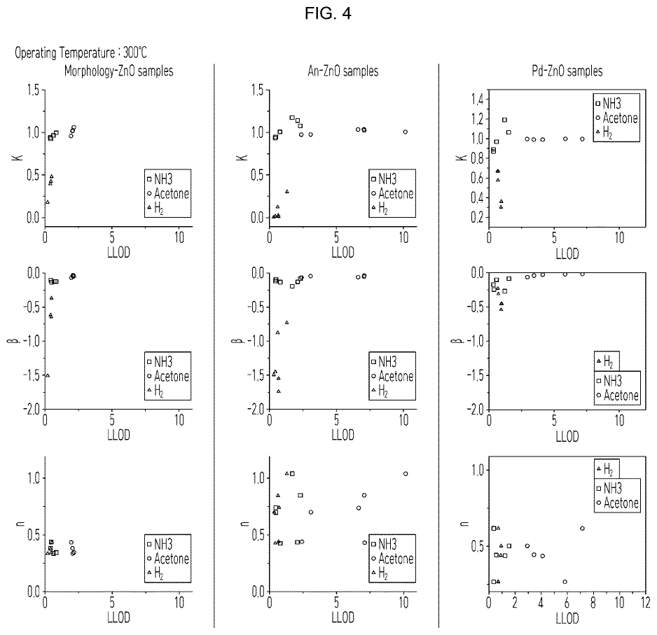

FIG. 4 to FIG. 7 show the results of graphing, as a scatter plot, data from three repeated measurements of the correlation between K and LLOD, the correlation between β (Beta) and LLOD, and the correlation between n and LLOD for each temperature (300° C., 350° C., 400° C., and 450° C.) for NH₃, H₂, and Acetone.

FIG. 4 shows data from three repeated measurements of the correlation of K&LLOD, Beta&LLOD, and n&LLOD parameters for NH₃, H₂ and Acetone gases at 300° C. using ZnO, Au—ZnO, and Pd—ZnO sensors, represented as a scatter plot. As shown in FIG. 4, although there were slight differences for each sensor, the distribution of points for each gas formed a certain pattern, such as being clustered in a certain coordinate group or forming a line. This pattern of FIG. 4 was confirmed to be formed differently for each gas.

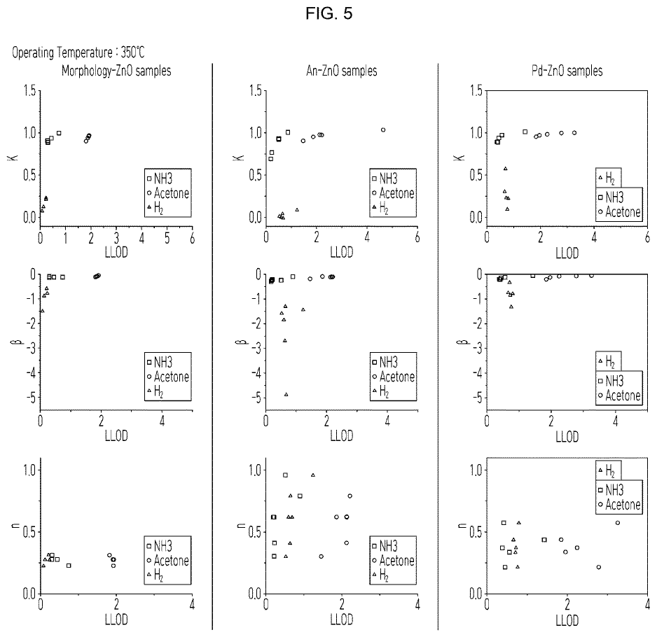

FIG. 5 shows data from three repeated measurements of the correlation of K&LLOD, Beta&LLOD, and n&LLOD parameters for NH₃, H₂ and Acetone gases at 350° C. using ZnO, Au—ZnO, and Pd—ZnO sensors, represented as a scatter plot. As shown in FIG. 5, although there were slight differences for each sensor, the distribution of points for each gas formed a certain pattern, such as being clustered in a certain coordinate group or forming a line. This pattern of FIG. 5 was formed differently for each gas. A point distribution of a similar pattern was confirmed to be formed compared to FIG. 4, although the position on the coordinates was different.

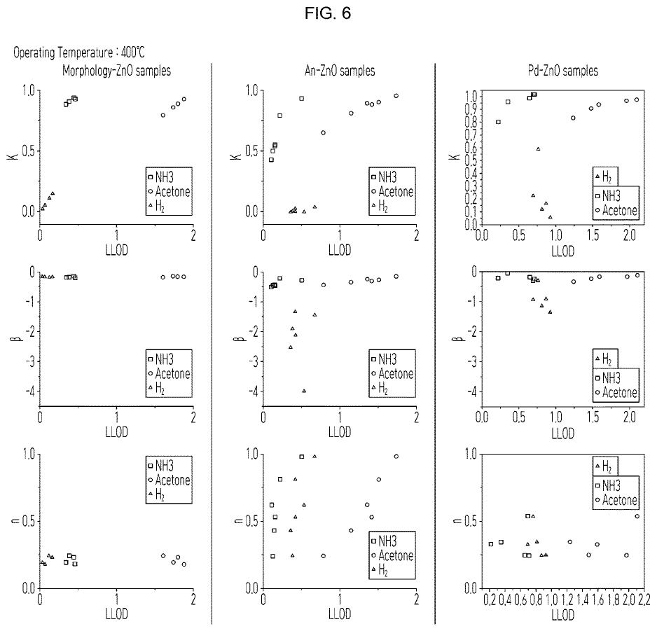

FIG. 6 shows data from three repeated measurements of the correlation of K&LLOD, Beta&LLOD, and n&LLOD parameters for NH₃, H₂ and Acetone gases at 400° C. using ZnO, Au—ZnO, and Pd—ZnO sensors, represented as a scatter plot. As shown in FIG. 6, although there were slight differences for each sensor, the distribution of points for each gas formed a certain pattern, such as being clustered in a certain coordinate group or forming a line. This pattern of FIG. 6 was formed differently for each gas. A point distribution of a similar pattern was confirmed to be formed compared to FIG. 4 and FIG. 5, although the position on the coordinates was different.

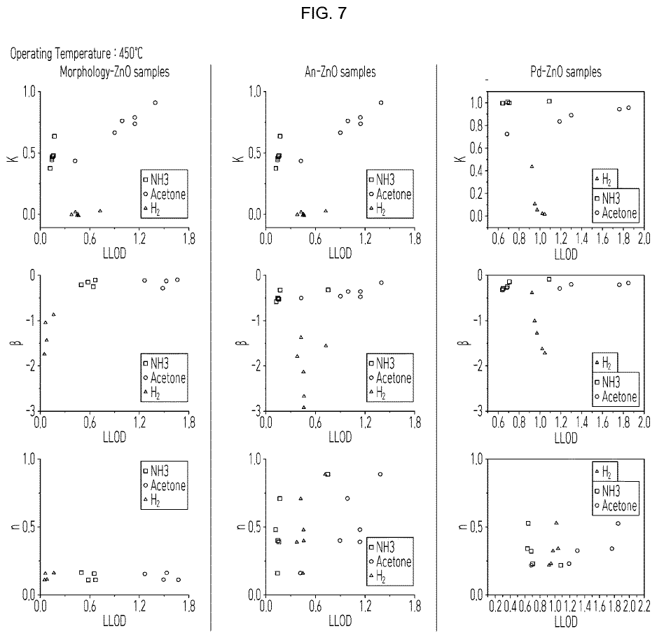

FIG. 7 shows data from three repeated measurements of the correlation of K&LLOD, Beta&LLOD, and n&LLOD parameters for NH₃, H₂ and Acetone gases at 450° C. using ZnO, Au—ZnO, and Pd—ZnO sensors, represented as a scatter plot. As shown in FIG. 7, although there were slight differences for each sensor, the distribution of points for each gas formed a certain pattern, such as being clustered in a certain coordinate group or forming a line. This pattern of FIG. 7 was formed differently for each gas. A point distribution of a similar pattern was confirmed to be formed compared to FIG. 4 to FIG. 6, although the position on the coordinates was different.

Therefore, by establishing as a standard the correlation between K and LLOD, the correlation between β (Beta) and LLOD, and the correlation between n and LLOD for each type of gas (NH₃, H₂ and Acetone) according to FIG. 4 to FIG. 7, and in the future, by measuring the correlation between K and LLOD, the correlation between β (Beta) and LLOD, and the correlation between n and LLOD for an external gas and comparing the results with the standard correlations of the parameters, if mutually similar results are obtained, the type of gas (NH₃, H₂ and Acetone) may be distinguished.

What is claimed is:

1. A gas sensing method comprising: i) capturing an external gas; ii) measuring a resistance value (Rₒ) of a gas sensor under a base gas condition to stabilize the gas sensor; iii) measuring resistance values (R_{g}) of the gas sensor under two or more temperature conditions with different temperatures by changing a temperature of the external gas; iv) calculating ratios (R_{g}/Rₒ) of the resistance value of the gas sensor under the base gas condition to the resistance values of the gas sensor under the two or more temperature conditions with different temperatures, and then calculating two or more kinetic parameters or static parameters from the ratios (R_{g}/Rₒ); and v) determining a type of the external gas by comparing a correlation of the two or more kinetic or static parameters calculated in the operation iv) with a standard correlation of two or more kinetic or static parameters.

2. The gas sensing method of claim 1, wherein the changing of the temperature of the captured external gas in operation iii) comprises heating or cooling the captured external gas using a temperature control device.

3. The gas sensing method of claim 2, wherein the temperature control device is a heater or a cooler.

4. The gas sensing method of claim 1, wherein the measuring of the resistance values (Rg) in the operation iii) further comprises measuring resistance values of the gas sensor under a condition wherein at least one selected from the group consisting of concentration, humidity, and light absorbance is different, by changing at least one selected from the group consisting of concentration, humidity, and light absorbance for a test sample.

5. The gas sensing method of claim 4, wherein the concentration, humidity, and light absorbance for the test sample are respectively controlled using (a) one of a gas dilution device or a gas concentration device, (b) a humidity control device, and (c) a light source device, installed inside or outside the gas sensor.

6. The gas sensing method of claim 1, wherein in operations ii) and iii), the gas sensor comprises a plurality of gas sensors arranged to detect a different gas, respectively, to simultaneously measure resistance values for a plurality of gases.

7. The gas sensing method of claim 1, wherein in the operation iv), the kinetic parameter is at least one selected from the group consisting of K, β (Beta), LLOD (lower limit of detection), Eb (Energy barrier), and n (Power law Exponent), and the static parameter is at least one selected from the group consisting of t90, T90, Carrier Concentration, and Oxygen Vacancy.

8. The gas sensing method of claim 1, wherein in the operation v), the correlation of the kinetic parameters is at least one selected from the group consisting of a correlation between K and LLOD, a correlation between β (Beta) and LLOD, and a correlation between n and LLOD.

9. The gas sensing method of claim 1, wherein in the operation v), the standard correlation of two or more kinetic or static parameters is data obtained from a correlation of two or more kinetic or static parameters of a target gas, calculated by repeating operations i) to iv) multiple times for the target gas to be measured.

10. The gas sensing method of claim 1, wherein in the operation v), the correlation of the two or more kinetic or static parameters is visualized as a scatter plot and then compared with a standard scatter plot representing the standard correlation of two or more kinetic or static parameters.
